(19) United States
(12) Patent Application Publication
(10) Pub. No.: US 2026/0171224 A1
(43) Pub. Date: Jun. 18, 2026

(54) TRANSITIONAL CARE METHOD AND SYSTEM FOR CHILDREN WITH LIVER DISEASES, AND STORAGE MEDIUM

(71) Applicant: SHENZHEN THIRD PEOPLE'S HOSPITAL (SHENZHEN INSTITUTE OF HEPATOLOGY), Shenzhen, Guangdong (CN)

(72) Inventors: Lulu Sun, Shenzhen, Guangdong (CN); Hongzhou Lu, Shenzhen, Guangdong (CN); Yingxia Liu, Shenzhen, Guangdong (CN); Jing Yuan, Shenzhen, Guangdong (CN); Xiaoming Ben, Shenzhen, Guangdong (CN); Tetsuya Asakawa, Shizuoka (JP); Yan Xu, Shenzhen, Guangdong (CN)

(21) Appl. No.: 18/711,928

(22) PCT Filed: Nov. 17, 2022
(86) PCT No.: PCT/CN2022/132684

§ 371 Date: May 21, 2024

(30) Foreign Application Priority Data
Nov. 23, 2021 (CN) 202111397715.1

(51) Int. Cl. G16H 40/20; G09B 5/06; G16H 30/40

(52) U.S. Cl. CPC G16H 40/20; G09B 5/065; G16H 30/40

(57) ABSTRACT

A transitional care method for children with liver diseases comprises the following steps: investigating readiness for hospital discharge of a patient to obtain an investigation result on the readiness for hospital discharge; judging whether transitional care is necessary according to the investigation result on the readiness for hospital discharge; obtaining body weight, height and a photo of the patient; obtaining a skin color and a sclera color of the patient according to the photo; judging whether the food eaten by the patient is edible; medical personnel conducting a consultation according to the body weight, the height, the skin color and the sclera color of the patient, the food eaten by the patient and an assessment result on health education knowledge, and providing a corresponding care plan; and sending the care plan to the patient or a guardian of the patient.

### TECHNICAL FIELD

The present invention relates to the technical field of medical care, and in particular to a transitional care method and a transitional care system for children with liver diseases, and a storage medium.

### BACKGROUND ART

Patients with chronic liver diseases need to be cared for after hospital discharge, and only careful and meticulous care can help the patients with liver diseases recover well. Therefore, the patients with liver diseases hope that the hospital can continue to provide medical care services after hospital discharge. Current transitional care services provided by hospitals are generally provided through manual follow-up, the transitional care efficiency is low, the effect is poor, and the degree of recognition in patients is low.

### SUMMARY OF THE PRESENT INVENTION

A main object of the present invention is to provide a transitional care method and a transitional care system for children with liver diseases, and a storage medium, aiming to solve the problems in the prior art that transitional care services are provided through manual follow-up, the transitional care efficiency is low, the effect is poor and the degree of recognition in patients is low.

In order to achieve the above object, the present invention provides a transitional care method for children with liver diseases, comprising the following steps:
investigating readiness for hospital discharge of a patient to obtain an investigation result on the readiness for hospital discharge;
judging whether transitional care is necessary according to the investigation result on the readiness for hospital discharge, and if the transitional care is necessary, performing the transitional care according to the following steps:
obtaining body weight and height of the patient within a first time period; photographing the patient within the first time period to obtain a photo of the patient; and obtaining a skin color and/or a sclera color of the patient according to the photo;
installing a monitoring device on the patient to recognize food eaten by the patient in real time, and judging whether the food eaten by the patient is edible; and if the food is not edible, the monitoring device sending out reminding information that the food is not edible;
issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge;
medical personnel conducting a consultation according to the body weight of the patient and/or the height of the patient and/or the skin color of the patient and/or the sclera color of the patient and/or the food eaten by the patient and/or the assessment result on the health education knowledge, and providing a corresponding care plan; and
sending the care plan to the patient and/or the guardian of the patient.

Optionally, the investigation on the readiness for hospital discharge comprises:
an investigation on discharge preparation and/or an investigation on quality of discharge teaching and/or an investigation on social support and/or an investigation on anxiety.

Optionally, the step of photographing the patient within the first time period to obtain the photo of the patient comprises the following steps:
photographing to obtain at least one first image including the skin of the patient within the first time period; and/or photographing to obtain at least one second image including the sclera of the patient within the first time period.

Optionally, the step of obtaining the skin color and/or the sclera color of the patient according to the photo comprises the following steps:
processing the first image and/or the second image based on a first preset network model to obtain an image type;
selecting a second preset network model according to the image type and processing the first image to obtain the skin color of the patient; and
selecting the second preset network model according to the image type and processing the second image to obtain the sclera color of the patient.

Optionally, the step of installing the monitoring device on the patient to recognize the food eaten by the patient in real time and judging whether the food eaten by the patient is edible comprises the following steps:
installing the monitoring device on the patient to obtain a video image of the food eaten by the patient in real time;
processing the video image based on a third preset network model to obtain a type of the food eaten by the patient; and
judging whether the food type is edible according to recipe information preset in the monitoring device; if the food type is within the preset recipe information, judging that the food type is edible correspondingly; and if the food type is not within the preset recipe information, judging that the food type is not edible correspondingly.

Optionally, the method further comprises the following steps:
installing the monitoring device on the patient to obtain an amount of exercise of the patient; and
judging whether the amount of exercise exceeds a threshold, and if the amount of exercise exceeds the threshold, the monitoring device sending out reminding information that the amount of exercise exceeds the threshold.

In addition, in order to achieve the above object, the present invention also provides a transitional care system for children with liver diseases, comprising a server, a monitoring device, a medical terminal and a patient terminal; wherein
the server is used for investigating readiness for hospital discharge of a patient to obtain an investigation result on the readiness for hospital discharge;
the server is further used for judging whether transitional care is necessary according to the investigation result on the readiness for hospital discharge;
the server is further used for obtaining a skin color and/or a sclera color of the patient according to a photo;
the server is further used for issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge;
the server is further used for sending a care plan to the patient and/or the guardian of the patient;
the monitoring device is used for recognizing food eaten by the patient in real time, judging whether the food eaten by the patient is edible, and sending out reminding information that the food is not edible if the food is not edible;
the monitoring device is further used for obtaining an amount of exercise of the patient, judging whether the amount of exercise exceeds a threshold, and sending out reminding information that the amount of exercise exceeds the threshold if the amount of exercise exceeds the threshold;
the patient terminal is used for obtaining body weight and height of the patient within a first time period, and photographing the patient within the first time period to obtain the photo of the patient; and
the medical terminal is used for medical personnel to conduct a consultation according to the body weight of the patient and/or the height of the patient and/or the skin color of the patient and/or the sclera color of the patient and/or the food eaten by the patient and/or the assessment result on the health education knowledge and provide the corresponding care plan.

Optionally, the server comprises:
a patient investigation module for investigating readiness for hospital discharge of a patient to obtain an investigation result on the readiness for hospital discharge;
a care judgment module for judging whether transitional care is necessary according to the investigation result on the readiness for hospital discharge;
a color obtaining module for obtaining a skin color and/or a sclera color of the patient according to a photo;
a training assessment module for issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge; and
a plan sending module for sending a care plan to the patient and/or the guardian of the patient.

Optionally, the monitoring device comprises:
a food judgment module for recognizing food eaten by the patient in real time, judging whether the food eaten by the patient is edible, and sending out reminding information that the food is not edible if the food is not edible; and
a judgment module on an amount of exercise for obtaining an amount of exercise of the patient and judging whether the amount of exercise exceeds a threshold, wherein the monitoring device sends out reminding information that the amount of exercise exceeds the threshold if the amount of exercise exceeds the threshold.

In addition, in order to achieve the above object, the present invention further provides a computer-readable storage medium, on which a computer program is stored, wherein, the computer program is executed by a processor to implement the above transitional care method for children with liver diseases.

From the above technical methods, the application has the following advantages:
according to the present invention, complete individual cases of patients with chronic liver diseases are achieved through data collection, and a transitional care pathway is dynamically formulated with condition changes, thereby improving the conditions of the patients with chronic liver diseases such as loss to follow-up, poor medication compliance, insufficient nutrition supply after hospital discharge and lagging growth and development.

### BRIEF DESCRIPTION OF THE DRAWINGS

FIG. 1 is a flow diagram of a transitional care method for children with liver diseases provided by the present invention.

FIG. 2 is a flow diagram of a color obtaining method provided by the present invention.

FIG. 3 is a flow diagram of a food recognition method provided by the present invention.

FIG. 4 is a flow diagram of a judgment method on an amount of exercise provided by the present invention.

FIG. 5 is a structural block diagram of an embodiment of a server according to the present invention.

FIG. 6 is a structural block diagram of an embodiment of a monitoring device according to the present invention.

FIG. 7 is a structural diagram of an embodiment of a transitional care system for children with liver diseases according to the present invention.

FIG. 8 is a structural diagram of a hardware operating environment involved in a plan of an embodiment of the present invention.

The implementation of the object, functional characteristics and advantages of the present invention are further described with reference to the embodiments and the drawings.

### EMBODIMENTS

In order to make the technical problems to be solved by the present invention, technical plans and beneficial effects thereof clearer and more understandable, the present invention is further described in detail below in conjunction with the drawings and the embodiments. It shall be understood that the specific embodiments described herein are only to explain the present invention, and are not used to limit the present invention.

In subsequent description, the use of suffixes such as “modules”, “components” or “units” for denoting elements is only for the benefit of description of the present invention, and they do not have specific meanings in themselves. Therefore, the “modules”, “components” or “units” can be used in a mixed manner.

It shall be noted that the terms “first”, “second”, etc. in the specification, the claims and the above drawings of the present invention are used to distinguish similar objects, and are not necessary to describe a specific order or sequence.

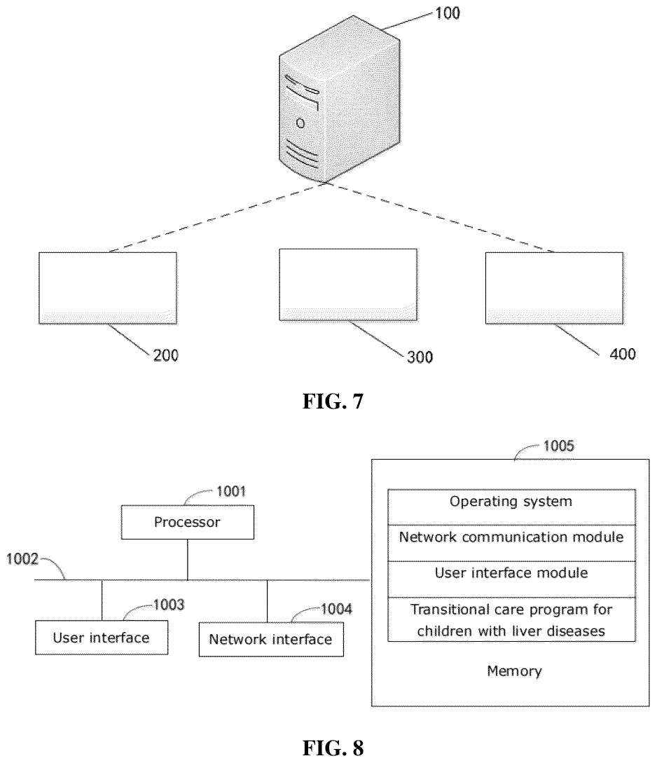

It shall be understood that the application is applied to a transitional care system for children with liver diseases. With reference to FIG. 7, FIG. 7 is a structural diagram of an embodiment of a transitional care system for children with liver diseases in an embodiment of the application, and as shown in FIG. 7, the system in FIG. 7 comprises a server 100, a monitoring device 200, a medical terminal 300 and a patient terminal 400; wherein
the server 100 is used for investigating readiness for hospital discharge of a patient to obtain an investigation result on the readiness for hospital discharge;
the server 100 is further used for judging whether transitional care is necessary according to the investigation result on the readiness for hospital discharge;
the server 100 is further used for obtaining a skin color and/or a sclera color of the patient according to a photo;
the server 100 is further used for issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge;
the server 100 is further used for sending a care plan to the patient and/or the guardian of the patient;
the monitoring device 200 is used for recognizing food eaten by the patient in real time, judging whether the food eaten by the patient is edible, and sending out reminding information that the food is not edible if the food is not edible;
the monitoring device 200 is further used for obtaining an amount of exercise of the patient, judging whether the amount of exercise exceeds a threshold, and sending out reminding information that the amount of exercise exceeds the threshold if the amount of exercise exceeds the threshold;
the patient terminal 400 is used for obtaining body weight and height of the patient within a first time period, and photographing the patient within the first time period to obtain the photo of the patient; and
the medical terminal 300 is used for medical personnel to conduct a consultation according to the body weight of the patient and/or the height of the patient and/or the skin color of the patient and/or the sclera color of the patient and/or the food eaten by the patient and/or the assessment result on the health education knowledge and provide the corresponding care plan.

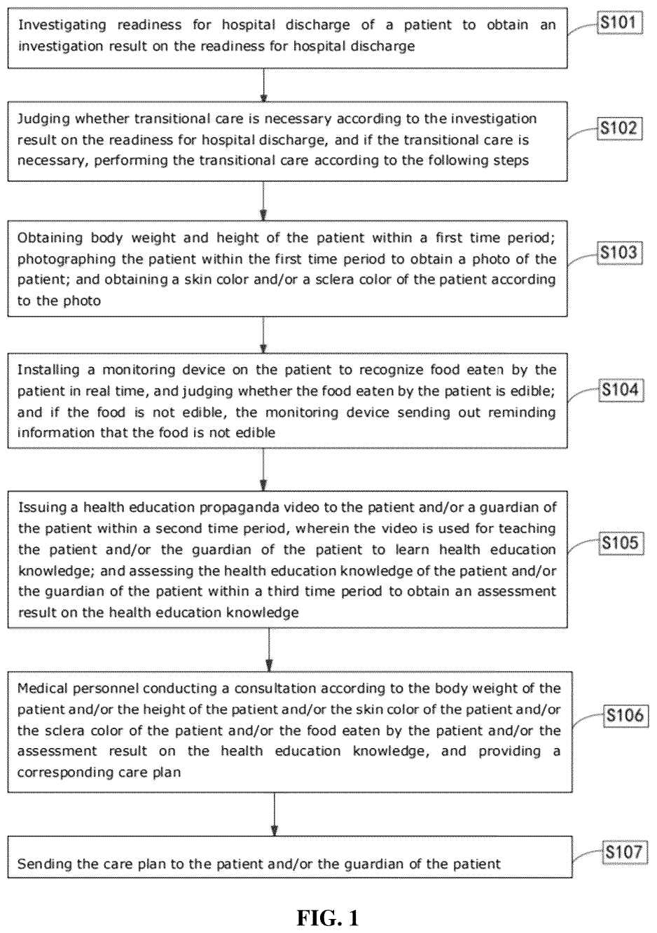

In an embodiment, as shown in FIG. 1, the present invention provides a transitional care method for children with liver diseases, comprising the following steps:

S101: investigating readiness for hospital discharge of a patient to obtain an investigation result on the readiness for hospital discharge.

When the children with liver diseases are ready for hospital discharge at the end of treatment, an investigation on the readiness for hospital discharge needs to be completed. The investigation on the readiness for hospital discharge is mainly carried out in a form of questionnaires. The investigation on the readiness for hospital discharge comprises an investigation on discharge preparation and/or an investigation on quality of discharge teaching and/or an investigation on social support and/or an investigation on anxiety. Each investigation corresponds to a questionnaire, for example, Readiness for Hospital Discharge Scale (RHDS), Quality of Discharge Teaching Scale (QDTS), Social Support Rating Scale (SSRS), Self-rating Anxiety Scale (SAS).

These questionnaires can be completed online, and a patient or a family member thereof submits predesigned relevant information at predetermined time, for example, body weight, etc.

Specific contents of the questionnaires are not defined in the technical plan, and the corresponding investigation contents can be designed according to specific demands.

S102: judging whether transitional care is necessary according to the investigation result on the readiness for hospital discharge, and if the transitional care is necessary, performing the transitional care according to the following steps.

A back-end server generates a corresponding investigation report for medical personnel to use according to the questionnaire provided by the patient or the family member thereof. The medical personnel judge whether the patient needs the transitional care according to the investigation report.

If the medical personnel find that some conditions of the current patient cannot meet requirements of the transitional care according to the investigation report, the medical personnel carry out special teaching for the patient or the family member thereof or contact a corresponding community or volunteer for help, etc. according to the investigation result; through the above teaching, after the patient meets the requirements of the transitional care after hospital discharge, the transitional care is allowed to be performed on the patient.

If the medical personnel judge that the transitional care after hospital discharge is allowed to be performed on the patient according to the investigation result, the transitional care is performed on the patient according to subsequent steps.

S103: obtaining body weight and height of the patient within a first time period; photographing the patient within the first time period to obtain a photo of the patient; and obtaining a skin color and/or a sclera color of the patient according to the photo.

The family member of the patient obtains physiological data of the patient such as body weight and height within a period, for example, every week or every day, and then reports the physiological data to the back-end server through an APP on a mobile phone. The obtained physiological data is as shown in the following table:

| | | |
|---|---|---|
| Height | 90 | cm |
| Body weight | 30.2 | kg |

The family member of the patient photographs to obtain at least one first photo (that is, a first image) including the skin of the patient and photographs to obtain at least one second photo (that is, a second image) including the sclera of the patient within a period, for example, every week or every day.

The family member uses the mobile phone to photograph the skin of the patient, such as the skin on faces and hands. Multiple photos including the skin of the patient can be obtained and uploaded to the server; and the mobile phone is used to photograph the sclera of the patient to obtain multiple photos including the sclera of the patient. The transitional care plan teaches the family member of the patient how to photograph the skin and sclera of the patient, and a photographing order is as follows: left hand->right hand->left face->front face->right face->sclera of left eye->sclera of right eye. The obtained photos are as shown in the following table:

| Nos. | Photo types | Photo names |
|---|---|---|
| 1 | Left hand | Left hand photo 1.jpg |
| 2 | Right hand | Right hand photo 2.jpg |
| 3 | Left face | Left face photo 1.jpg |
| 4 | Front face | Front face photo 2.jpg |
| 5 | Right face | Right face photo 1.jpg |
| 6 | Sclera of left eye | Sclera-of-left-eye photo 1.jpg |
| 7 | Sclera of right eye | Sclera-of-right-eye photo 2.jpg |

When the photos are uploaded through the APP on the mobile phone, the photo types are uploaded for the server to perform corresponding processing according to the photo types.

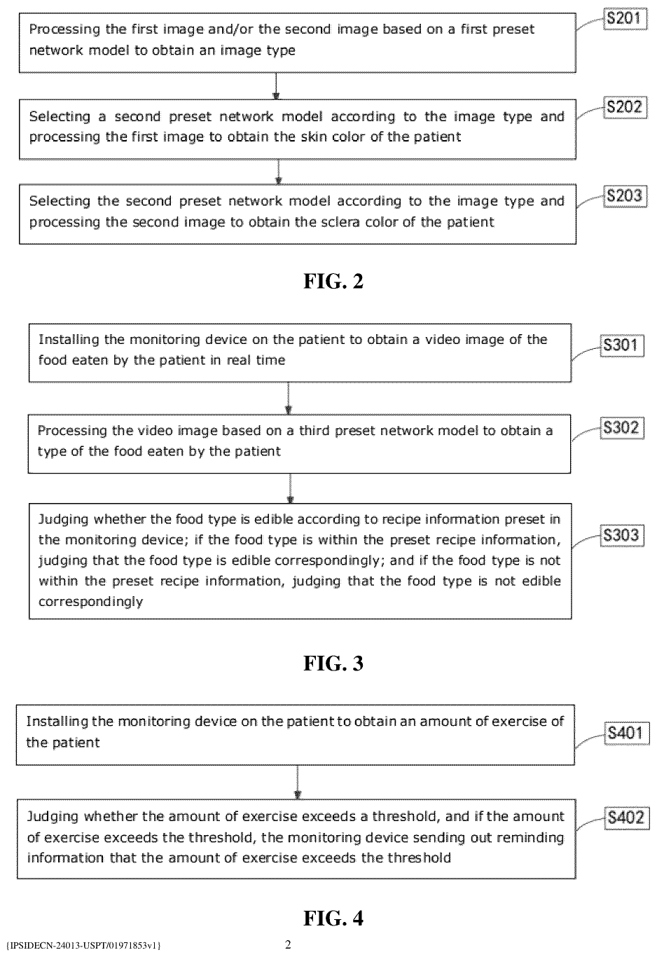

After the back-end server receives the skin and sclera photos of the patient, the corresponding photos are obtained according to the photo types. The corresponding photos are input into a preset network model to judge skin and sclera colors of the patient. A specific flow is as shown in a flow in FIG. 2.

S201: processing the first image and/or the second image based on a first preset network model to obtain an image type.

The first preset network model is deployed on the server, and the first preset network model may be various types of network models that can extract image features. For example, the first preset network model may refer to a U-Net (U-Network) model, a PSPNet (Pyramid Scene Parsing Network) model, a DenseNet (Dense Convolutional Network) model, a ResNet (Residual Network) model or a MobileNet (Mobile Network) model, etc. The first preset network model itself can have initial parameters, and the initial parameters may be parameters pre-trained on an ImageNet dataset.

In a first iterative training process, the first preset network model is trained on the basis of the initial parameters.

A monitored model training method is used in the embodiment of the application, and all sample images have marks for marking real recognition results of the sample images. In the embodiment of the application, the recognition results of the sample images include image type results.

Classifying and marking are performed accordingly. The image types include left hand, right hand, left face, front face, right face, sclera of left eye and sclera of right eye; and the image types may also be other types, for example, hand, face and sclera. Specific classification of the images can be set according to actual conditions, which is not limited in the technical plan. The technical plan is described by taking the image types of hand, face and sclera as an example.

The images corresponding to the image types are obtained according to the image types, including the images of the hand, the face and the sclera. These images are marked, and an attribution type of each image is marked. Then these marked images are used to train the first preset network model to obtain the trained first preset network model.

The first preset network model processes the images reported by the patient or the family member of the patient through the APP to obtain an image type, and if the obtained image type is not one of the hand, the face and the sclera, the image type appointed when the images are reported through the APP will prevail.

S202: selecting a second preset network model according to the image type and processing the first image to obtain the skin color of the patient.

S203: selecting the second preset network model according to the image type and processing the second image to obtain the sclera color of the patient.

The second preset network model may be a convolutional neural network or a deep learning neural network. The specific neural network is not limited in the embodiment.

Hand, face and sclera images for each image type are obtained, some of these images are from normal people, and some images are from patients with liver diseases. These images are marked, and then the marked images are used to train the second preset network model. A trained second preset network model is obtained for each classification, which is as described in the following table:

| Classification identifiers | Second preset network models |
|---|---|
| Hand | Hand-network model |
| Face | Face-network model |
| Sclera | Sclera-network model |

After the image type (for example, the hand) corresponding to the images reported by the patient or the family member of the patient through the APP is recognized according to the first preset network model, the trained second preset network model (for example, the hand-network model) corresponding to the type is obtained to process the images.

After the hand-network model detects the images, the identifiers including a hand color of the patient and a color depth are obtained.

| Photo name | Hand color | Color depth |
|---|---|---|
| Left hand photo 1.jpg | Yellow | Deep yellow |

After the second preset network model processes the images reported by the patient or the family member of the patient through the APP, the detection results as shown in the following table are obtained:

| | Photo | | | Color |
|---|---|---|---|---|
| Nos. | types | Photo names | Colors | depths |
| 1 | Hand | Left hand photo 1.jpg | Yellow | Deep yellow |
| 2 | Hand | Right hand photo 2.jpg | Yellow | Deep yellow |
| 3 | Face | Left face photo 1.jpg | Yellow | Deep yellow |
| 4 | Face | Front face photo 2.jpg | Yellow | Deep yellow |
| 5 | Face | Right face photo 1.jpg | Yellow | Deep yellow |
| 6 | Sclera | Sclera-of-left-eye photo 1.jpg | White | Light white |
| 7 | Sclera | Sclera-of-right-eye photo 2.jpg | White | Light white |

S104: installing a monitoring device on the patient to recognize food eaten by the patient in real time, and judging whether the food eaten by the patient is edible; and if the food is not edible, the monitoring device sending out reminding information that the food is not edible.

The monitoring device is installed on the patient with a liver disease, for example, a smart device such as a smart watch, a smart bracelet or a smart badge. Before the patient eats, the monitoring device obtains an image content of food and then uploads the image content to the back-end server.

The back-end server recognizes the image according to the image including the food and obtained by the smart device of the patient to obtain the type of the food eaten by the patient. See a flow shown in FIG. 3 for details:

S301: installing the monitoring device on the patient to obtain a video image of the food eaten by the patient in real time.

Before the patient eats, the monitoring device obtains an image content of food and then uploads the image content to the back-end server.

S302: processing the video image based on a third preset network model to obtain a type of the food eaten by the patient.

The third preset network model may be a convolutional neural network or a deep learning neural network. The specific neural network is not limited in the embodiment.

The images including the food are obtained, some of the food included in these images is edible for the patient with the liver disease, and some of the food is not edible. These images are marked, and then the marked images are used to train the third preset network model.

The images reported by the smart device (that is, the monitoring device) of the patient are processed by a trained third preset network model to obtain the corresponding food type, as shown in the following table:

| Photo name | Food type |
|---|---|
| Food 1.jpg | Apple |

The server returns the recognized food type to the smart device of the patient.

S303: judging whether the food type is edible according to recipe information preset in the monitoring device; if the food type is within the preset recipe information, judging that the food type is edible correspondingly; and if the food type is not within the preset recipe information, judging that the food type is not edible correspondingly.

The server will issue the edible recipe information of the patient to the monitoring device of the patient according to a condition of the patient. For example, the recipe information is as shown in the following table:

Food types

Food 1
Food 2
Food 3

After receiving the food type returned by the server, the monitoring device (for example, a smart wearable device) of the patient judges whether the food is edible according to the edible recipe information saved locally. If the food is not edible, for example, if the server detects that the type of the food that the patient wants to eat is fried food such as deep-fried dough sticks, the monitoring device sends out reminding information to remind the patient that the food is not edible. The reminding can be carried out through text display or voice playing.

S105: issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge.

The server issues health education propaganda videos to the APP on the mobile phone of the patient or the guardian thereof regularly (for example, every week), and the patient or the guardian thereof learns how to care for the patient with the liver disease according to these health education propaganda videos in the APP. At the same time, an assessment on relevant knowledge of the patient or the guardian thereof is carried out regularly according to a learning result, and an assessment result is saved on the server for subsequent use by medical personnel. For example, an assessment on relevant knowledge of a child aged 7 years or older and a parent of a child under 7 years old is carried out.

S106: medical personnel conducting a consultation according to the body weight of the patient and/or the height of the patient and/or the skin color of the patient and/or the sclera color of the patient and/or the food eaten by the patient and/or the assessment result on the health education knowledge, and providing a corresponding care plan.

S107: sending the care plan to the patient and/or the guardian of the patient.

The medical personnel (such as nurses) conduct a consultation according to the body weight, height, skin color and sclera color of the patient, the food eaten by the patient and the assessment result on the health education knowledge that are collected in the server. For example, a network professional nurse group is organized to conduct the consultation, and the care plan corresponding to the patient is provided.

After the nurses conduct the consultation, the care plan is uploaded to the server through the APP on the mobile phone, and the server forwards the consultation plan to the APP on the mobile phone of the patient or the guardian thereof.

After the APP on the mobile phone of the patient or the guardian thereof receives the care plan issued by the server, the patient is cared for according to the care plan.

According to the present invention, complete individual cases of patients with chronic liver diseases are achieved through data collection, and a transitional care pathway is dynamically formulated with condition changes, thereby improving the conditions of the patients with chronic liver diseases such as loss to follow-up, poor medication compliance, insufficient nutrition supply after hospital discharge and lagging growth and development.

In addition, an embodiment of the present invention further proposes another transitional care method for children with liver diseases. With reference to FIG. 4, the method further comprises the following steps in the method of FIG. 1:

S401: installing the monitoring device on the patient to obtain an amount of exercise of the patient.

The monitoring device is installed on the patient with a liver disease, for example, a smart device such as a smart watch, a smart bracelet or a smart badge. The mount of exercise of the patient is counted in real time. For example, steps of the patient are counted to count the daily steps of the patient.

S402: judging whether the amount of exercise exceeds a threshold, and if the amount of exercise exceeds the threshold, the monitoring device sending out reminding information that the amount of exercise exceeds the threshold.

The server will issue an allowable amount of exercise for the patient according to a condition of the patient. For example, the daily amount of exercise does not exceed 1000 steps. The specific amount of exercise is dynamically adjusted according to the condition.

After obtaining the amount of exercise of the patient, the monitoring device (for example, the smart bracelet) of the patient judges whether the amount of exercise today exceeds the allowable amount of exercise for the patient. If the amount of exercise today exceeds the allowable amount of exercise, the monitoring device reminds the patient to reduce exercise. The reminding can be carried out through text display or voice playing.

According to the present invention, complete individual cases of patients with chronic liver diseases are achieved through data collection, and a transitional care pathway is dynamically formulated with condition changes, thereby improving the conditions of the patients with chronic liver diseases such as loss to follow-up, poor medication compliance, insufficient nutrition supply after hospital discharge and lagging growth and development.

In addition, an embodiment of the present invention further proposes a transitional care system for children with liver diseases. With reference to FIG. 7, the system comprises a server 100, a monitoring device 200, a medical terminal 300 and a patient terminal 400; wherein
the server 100 is used for investigating readiness for hospital discharge of a patient to obtain an investigation result on the readiness for hospital discharge;
the server 100 is further used for judging whether transitional care is necessary according to the investigation result on the readiness for hospital discharge;
the server 100 is further used for obtaining a skin color and/or a sclera color of the patient according to a photo;
the server 100 is further used for issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge;
the server 100 is further used for sending a care plan to the patient and/or the guardian of the patient;
the monitoring device 200 is used for recognizing food eaten by the patient in real time, judging whether the food eaten by the patient is edible, and sending out reminding information that the food is not edible if the food is not edible;
the monitoring device 200 is further used for obtaining an amount of exercise of the patient, judging whether the amount of exercise exceeds a threshold, and sending out reminding information that the amount of exercise exceeds the threshold if the amount of exercise exceeds the threshold;
the patient terminal 400 is used for obtaining body weight and height of the patient within a first time period, and photographing the patient within the first time period to obtain the photo of the patient; and
the medical terminal 300 is used for medical personnel to conduct a consultation according to the body weight of the patient and/or the height of the patient and/or the skin color of the patient and/or the sclera color of the patient and/or the food eaten by the patient and/or the assessment result on the health education knowledge and provide the corresponding care plan.

According to the present invention, complete individual cases of patients with chronic liver diseases are achieved through data collection, and a transitional care pathway is dynamically formulated with condition changes, thereby improving the conditions of the patients with chronic liver diseases such as loss to follow-up, poor medication compliance, insufficient nutrition supply after hospital discharge and lagging growth and development.

In addition, an embodiment of the present invention further proposes a server. With reference to FIG. 5, the server 100 comprises:
a patient investigation module 101 for investigating readiness for hospital discharge of a patient to obtain an investigation result on the readiness for hospital discharge;
a care judgment module 102 for judging whether transitional care is necessary according to the investigation result on the readiness for hospital discharge;
a color obtaining module 103 for obtaining a skin color and/or a sclera color of the patient according to a photo;
a training assessment module 104 for issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge; and
a plan sending module 105 for sending a care plan to the patient and/or the guardian of the patient.

According to the present invention, complete individual cases of patients with chronic liver diseases are achieved through data collection, and a transitional care pathway is dynamically formulated with condition changes, thereby improving the conditions of the patients with chronic liver diseases such as loss to follow-up, poor medication compliance, insufficient nutrition supply after hospital discharge and lagging growth and development.

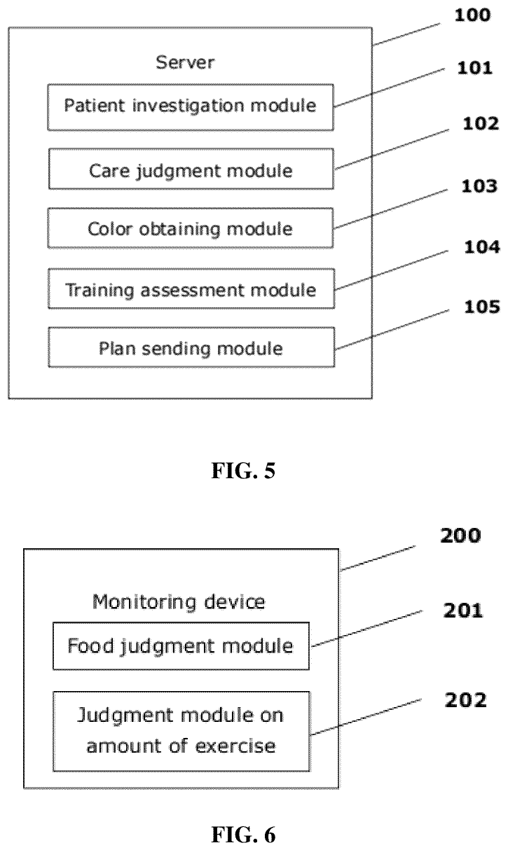

In addition, an embodiment of the present invention further proposes a monitoring device. With reference to FIG. 6, the monitoring device 200 comprises:
a food judgment module 201 for recognizing food eaten by the patient in real time, judging whether the food eaten by the patient is edible, and sending out reminding information that the food is not edible if the food is not edible; and
a judgment module on an amount of exercise 202 for obtaining an amount of exercise of the patient and judging whether the amount of exercise exceeds a threshold, wherein the monitoring device sends out reminding information that the amount of exercise exceeds the threshold if the amount of exercise exceeds the threshold.

According to the present invention, complete individual cases of patients with chronic liver diseases are achieved through data collection, and a transitional care pathway is dynamically formulated with condition changes, thereby improving the conditions of the patients with chronic liver diseases such as loss to follow-up, poor medication compliance, insufficient nutrition supply after hospital discharge and lagging growth and development.

With reference to FIG. 8, FIG. 8 is a structural diagram of a hardware operating environment of the server 100, the monitoring device 200, the medical terminal 300 and the patient terminal 400 involved in the plan of the embodiment of the present invention.

As shown in FIG. 8, the hardware operating environment may comprise: a processor 1001 such as a CPU, a communication bus 1002, a user interface 1003, a network interface 1004 and a memory 1005. The communication bus 1002 is used for achieving connection communication among these components. The user interface 1003 may comprise a display and an input unit such as a keyboard, and optionally, the user interface 1003 may also comprise standard wired and wireless interfaces. The network interface 1004 may optionally comprise standard wired and wireless interfaces (such as WI-FI, 4G and 5G interfaces). The memory 1005 may be a high-speed RAM or a non-volatile memory, such as a disk memory. The memory 1005 may optionally be a storage device independent of the foregoing processor 1001.

Those skilled in the art can understand that the structure shown in FIG. 8 does not constitute a limitation to the server 100, the monitoring device 200, the medical terminal 300 or the patient terminal 400, and can comprise components more or less than those shown in FIG. 8, or combine some components, or comprise different arrangements of components.

As shown in FIG. 8, the memory 1005 as a computer storage medium may comprise an operating system, a network communication module, a user interface module and a transitional care program for children with liver diseases.

In the hardware operating environment as shown in FIG. 8, the network interface 1004 is mainly used for performing data communication with an external network; the user interface 1003 is mainly used for receiving input instructions from a user; and in the hardware operating environment, the processor 1001 calls the transitional care program for children with liver diseases stored in the memory 1005, and executes the following operations:
investigating readiness for hospital discharge of a patient to obtain an investigation result on the readiness for hospital discharge;
judging whether transitional care is necessary according to the investigation result on the readiness for hospital discharge, and if the transitional care is necessary, performing the transitional care according to the following steps:
obtaining body weight and height of the patient within a first time period; photographing the patient within the first time period to obtain a photo of the patient; and obtaining a skin color and/or a sclera color of the patient according to the photo;
installing a monitoring device on the patient to recognize food eaten by the patient in real time, and judging whether the food eaten by the patient is edible; and if the food is not edible, the monitoring device sending out reminding information that the food is not edible;
issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge;
medical personnel conducting a consultation according to the body weight of the patient and/or the height of the patient and/or the skin color of the patient and/or the sclera color of the patient and/or the food eaten by the patient and/or the assessment result on the health education knowledge, and providing a corresponding care plan; and
sending the care plan to the patient and/or the guardian of the patient.

Optionally, the investigation on the readiness for hospital discharge comprises:
an investigation on discharge preparation and/or an investigation on quality of discharge teaching and/or an investigation on social support and/or an investigation on anxiety.

Optionally, the step of photographing the patient within the first time period to obtain the photo of the patient comprises the following steps:
photographing to obtain at least one first image including the skin of the patient within the first time period; and/or
photographing to obtain at least one second image including the sclera of the patient within the first time period.

Optionally, the step of obtaining the skin color and/or the sclera color of the patient according to the photo comprises the following steps:
processing the first image and/or the second image based on a first preset network model to obtain an image type;
selecting a second preset network model according to the image type and processing the first image to obtain the skin color of the patient; and
selecting the second preset network model according to the image type and processing the second image to obtain the sclera color of the patient.

Optionally, the step of installing the monitoring device on the patient to recognize the food eaten by the patient in real time and judging whether the food eaten by the patient is edible comprises the following steps:
installing the monitoring device on the patient to obtain a video image of the food eaten by the patient in real time;
processing the video image based on a third preset network model to obtain a type of the food eaten by the patient; and
judging whether the food type is edible according to recipe information preset in the monitoring device; if the food type is within the preset recipe information, judging that the food type is edible correspondingly; and if the food type is not within the preset recipe information, judging that the food type is not edible correspondingly.

Optionally, the method further comprises the following steps:
installing the monitoring device on the patient to obtain an amount of exercise of the patient; and
judging whether the amount of exercise exceeds a threshold, and if the amount of exercise exceeds the threshold, the monitoring device sending out reminding information that the amount of exercise exceeds the threshold.

According to the present invention, complete individual cases of patients with chronic liver diseases are achieved through data collection, and a transitional care pathway is dynamically formulated with condition changes, thereby improving the conditions of the patients with chronic liver diseases such as loss to follow-up, poor medication compliance, insufficient nutrition supply after hospital discharge and lagging growth and development.

In addition, an embodiment of the present invention further proposes a computer-readable storage medium, on which a transitional care program for children with liver diseases is stored, wherein, the transitional care program for children with liver diseases is executed by the processor to implement the following operations:
investigating readiness for hospital discharge of a patient to obtain an investigation result on the readiness for hospital discharge;
judging whether transitional care is necessary according to the investigation result on the readiness for hospital discharge, and if the transitional care is necessary, performing the transitional care according to the following steps:
obtaining body weight and height of the patient within a first time period; photographing the patient within the first time period to obtain a photo of the patient; and obtaining a skin color and/or a sclera color of the patient according to the photo;
installing a monitoring device on the patient to recognize food eaten by the patient in real time, and judging whether the food eaten by the patient is edible; and if the food is not edible, the monitoring device sending out reminding information that the food is not edible;
issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge;
medical personnel conducting a consultation according to the body weight of the patient and/or the height of the patient and/or the skin color of the patient and/or the sclera color of the patient and/or the food eaten by the patient and/or the assessment result on the health education knowledge, and providing a corresponding care plan; and
sending the care plan to the patient and/or the guardian of the patient.

Optionally, the investigation on the readiness for hospital discharge comprises:
an investigation on discharge preparation and/or an investigation on quality of discharge teaching and/or an investigation on social support and/or an investigation on anxiety.

Optionally, the step of photographing the patient within the first time period to obtain the photo of the patient comprises the following steps:
photographing to obtain at least one first image including the skin of the patient within the first time period; and/or photographing to obtain at least one second image including the sclera of the patient within the first time period.

Optionally, the step of obtaining the skin color and/or the sclera color of the patient according to the photo comprises the following steps:
processing the first image and/or the second image based on a first preset network model to obtain an image type;
selecting a second preset network model according to the image type and processing the first image to obtain the skin color of the patient; and
selecting the second preset network model according to the image type and processing the second image to obtain the sclera color of the patient.

Optionally, the step of installing the monitoring device on the patient to recognize the food eaten by the patient in real time and judging whether the food eaten by the patient is edible comprises the following steps:
installing the monitoring device on the patient to obtain a video image of the food eaten by the patient in real time;
processing the video image based on a third preset network model to obtain a type of the food eaten by the patient; and
judging whether the food type is edible according to recipe information preset in the monitoring device; if the food type is within the preset recipe information, judging that the food type is edible correspondingly; and if the food type is not within the preset recipe information, judging that the food type is not edible correspondingly.

Optionally, the method further comprises the following steps:
installing the monitoring device on the patient to obtain an amount of exercise of the patient; and
judging whether the amount of exercise exceeds a threshold, and if the amount of exercise exceeds the threshold, the monitoring device sending out reminding information that the amount of exercise exceeds the threshold.

According to the present invention, complete individual cases of patients with chronic liver diseases are achieved through data collection, and a transitional care pathway is dynamically formulated with condition changes, thereby improving the conditions of the patients with chronic liver diseases such as loss to follow-up, poor medication compliance, insufficient nutrition supply after hospital discharge and lagging growth and development.

It shall be noted that the terms “comprising”, “including” or any other variation thereof herein are intended to cover non-exclusive inclusions, so that a process, a method, an article or a system comprising a series of elements comprises not only those elements, but also other elements that are not explicitly listed, or elements that are inherent to the process, the method, the article or the system. In the absence of more limitations, an element defined by a statement “comprising a . . . ” does not preclude the existence of other identical elements in the process, the method, the article or the system comprising the element.

Serial numbers of the above embodiments in the present invention are only for description and do not represent the priority level of the embodiments.

Through the description of the above embodiments, those skilled in the art can clearly understand that the methods in the above embodiments can be implemented by virtue of software and a necessary general hardware platform, and naturally can also be implemented by virtue of hardware. However, under many circumstances, the former is a preferred embodiment. Based on this understanding, the technical plan of the present invention in essence or a part that contributes to the prior art may be reflected in the form of a software product, and the computer software product is stored in a storage medium (such as a ROM/RAM, a disk, an optical disk) as mentioned above, and comprises several instructions to enable a terminal device (which may be a mobile phone, a computer, a server, a controller or a network device, etc.) to execute the methods in various embodiments of the present invention.

The above contents are only preferred embodiments of the present invention, and do not limit the patent scope of the present invention. All equivalent structures or equivalent flow transformations made by using the contents of the specification and the drawings of the present invention, or directly or indirectly applied in other related technical fields, are similarly included in the patent protection scope of the present invention.

1. A transitional care method for children with liver diseases, comprising the following steps:
investigating readiness for hospital discharge of a patient to obtain an investigation result on the readiness for hospital discharge;
judging whether transitional care is necessary according to the investigation result on the readiness for hospital discharge, and if the transitional care is necessary, performing the transitional care according to the following steps:
obtaining body weight and height of the patient within a first time period; photographing the patient within the first time period to obtain a photo of the patient; and obtaining a skin color and/or a sclera color of the patient according to the photo;
installing a monitoring device on the patient to recognize food eaten by the patient in real time, and judging whether the food eaten by the patient is edible; and if the food is not edible, the monitoring device sending out reminding information that the food is not edible;
issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge;
medical personnel conducting a consultation according to the body weight of the patient and/or the height of the patient and/or the skin color of the patient and/or the sclera color of the patient and/or the food eaten by the patient and/or the assessment result on the health education knowledge, and providing a corresponding care plan; and
sending the care plan to the patient and/or the guardian of the patient.

2. The method according to claim 1, wherein, the investigation on the readiness for hospital discharge comprises:
an investigation on discharge preparation and/or an investigation on quality of discharge teaching and/or an investigation on social support and/or an investigation on anxiety.

3. The method according to claim 1, wherein, the step of photographing the patient within the first time period to obtain the photo of the patient comprises the following steps:
photographing to obtain at least one first image including the skin of the patient within the first time period; and/or
photographing to obtain at least one second image including the sclera of the patient within the first time period.

4. The method according to claim 3, wherein, the step of obtaining the skin color and/or the sclera color of the patient according to the photo comprises the following steps:
processing the first image and/or the second image based on a first preset network model to obtain an image type;
selecting a second preset network model according to the image type and processing the first image to obtain the skin color of the patient; and
selecting the second preset network model according to the image type and processing the second image to obtain the sclera color of the patient.

5. The method according to claim 1, wherein, the step of installing the monitoring device on the patient to recognize the food eaten by the patient in real time and judging whether the food eaten by the patient is edible comprises the following steps:
installing the monitoring device on the patient to obtain a video image of the food eaten by the patient in real time;
processing the video image based on a third preset network model to obtain a type of the food eaten by the patient; and
judging whether the food type is edible according to recipe information preset in the monitoring device; if the food type is within the preset recipe information, judging that the food type is edible correspondingly; and if the food type is not within the preset recipe information, judging that the food type is not edible correspondingly.

6. The method according to claim 1, wherein, the method further comprises the following steps:
installing the monitoring device on the patient to obtain an amount of exercise of the patient; and
judging whether the amount of exercise exceeds a threshold, and if the amount of exercise exceeds the threshold, the monitoring device sending out reminding information that the amount of exercise exceeds the threshold.

7. A transitional care system for children with liver diseases, comprising a server, a monitoring device, a medical terminal and a patient terminal; wherein
the server is used for investigating readiness for hospital discharge of a patient to obtain an investigation result on the readiness for hospital discharge;
the server is further used for judging whether transitional care is necessary according to the investigation result on the readiness for hospital discharge;
the server is further used for obtaining a skin color and/or a sclera color of the patient according to a photo;
the server is further used for issuing a health education propaganda video to the patient and/or a guardian of the patient within a second time period, wherein the video is used for teaching the patient and/or the guardian of the patient to learn health education knowledge; and assessing the health education knowledge of the patient and/or the guardian of the patient within a third time period to obtain an assessment result on the health education knowledge;
the server is further used for sending a care plan to the patient and/or the guardian of the patient;
the monitoring device is used for recognizing food eaten by the patient in real time, judging whether the food eaten by the patient is edible, and sending out reminding information that the food is not edible if the food is not edible;
the monitoring device is further used for obtaining an amount of exercise of the patient, judging whether the amount of exercise exceeds a threshold, and sending out reminding information that the amount of exercise exceeds the threshold if the amount of exercise exceeds the threshold;
the patient terminal is used for obtaining body weight and height of the patient within a first time period, and photographing the patient within the first time period to obtain the photo of the patient; and
the medical terminal is used for medical personnel to conduct a consultation according to the body weight of the patient and/or the height of the patient and/or the skin color of the patient and/or the sclera color of the patient and/or the food eaten by the patient and/or the assessment result on the health education knowledge and provide the corresponding care plan.

8. The system according to claim 7, wherein, the server comprises:
a patient investigation module for investigating readiness for hospital discharge of a patient to obtain an investigation result on the readiness for hospital discharge;
a care judgment module for judging whether transitional care is necessary according to the investigation result on the readiness for hospital discharge;
a color obtaining module for obtaining a skin color and/or a sclera color of the patient according to a photo;
a training assessment module for issuing a health education propaganda video to the patient and/or a guardian of the patient within a second time period, wherein the video is used for teaching the patient and/or the guardian of the patient to learn health education knowledge; and
assessing the health education knowledge of the patient and/or the guardian of the patient within a third time period to obtain an assessment result on the health education knowledge; and
a plan sending module for sending a care plan to the patient and/or the guardian of the patient.

9. The system according to claim 7, wherein, the monitoring device comprises:
a food judgment module for recognizing food eaten by the patient in real time, judging whether the food eaten by the patient is edible, and sending out reminding information that the food is not edible if the food is not edible; and
a judgment module on an amount of exercise for obtaining an amount of exercise of the patient and judging whether the amount of exercise exceeds a threshold, wherein the monitoring device sends out reminding information that the amount of exercise exceeds the threshold if the amount of exercise exceeds the threshold.

10. A computer-readable storage medium, on which a computer program is stored, wherein, the computer program is executed by a processor to implement the transitional care method for children with liver diseases according to claim 1.

11. A computer-readable storage medium, on which a computer program is stored, wherein, the computer program is executed by a processor to implement the transitional care method for children with liver diseases according to claim 2.

12. A computer-readable storage medium, on which a computer program is stored, wherein, the computer program is executed by a processor to implement the transitional care method for children with liver diseases according to claim 3.

13. A computer-readable storage medium, on which a computer program is stored, wherein, the computer program is executed by a processor to implement the transitional care method for children with liver diseases according to claim 4.

14. A computer-readable storage medium, on which a computer program is stored, wherein, the computer program is executed by a processor to implement the transitional care method for children with liver diseases according to claim 5.

15. A computer-readable storage medium, on which a computer program is stored, wherein, the computer program is executed by a processor to implement the transitional care method for children with liver diseases according to claim 6.
